(19) United States
(12) Patent Application Publication
(10) Pub. No.: US 2016/0174912 A1
(43) Pub. Date: Jun. 23, 2016

(54) LONG TERM HARM DETECTION WEARABLE DEVICE

(71) Applicant: KONINKLIJKE PHILIPS N.V., EINDHOVEN (NL)

(72) Inventor: John Cronin, Bonita Springs, FL (US)

(21) Appl. No.: 14/972,365

(22) Filed: Dec. 17, 2015

Related U.S. Application Data
(60) Provisional application No. 62/095,303, filed on Dec. 22, 2014.

(30) Foreign Application Priority Data
Jun. 11, 2015 (EP) 15171611.5

(51) Int. Cl. A61B 5/00; G01J 1/18; G08B 21/12; G01J 1/42

(52) U.S. Cl. CPC A61B 5/746; A61B 5/6801; A61B 5/742; A61B 5/0015; A61B 5/7282; G01J 1/429; G01J 1/18; G08B 21/12; A61B 2560/0242

(57) ABSTRACT

Systems and computer-implemented methods for detecting and informing a user about effects of long term exposures is provided. The method comprises updating information relating to one or more exposures to be detected from a network, wherein the updated information includes health-based threshold limits and is stored on a wearable device: obtaining sensor data from the various sensors associated with a wearable device, wherein the sensor data relates to one or more exposures to be detected; calculating a real-time exposure level based on the obtained sensor data; evaluating whether the calculated real-time exposure level exceeds health-based threshold limits; calculating an updated long-term exposure level, wherein the calculated long-term exposure level includes combining the calculated real-time exposure level with previously stored long-term exposure level calculations; evaluating whether the calculated updated long-term exposure level exceeds health-based threshold limits: and storing the calculated updated long-term exposure level into memory of the wearable device, wherein the stored calculated long-term exposure level is used to calculate a next updated long-term exposure level.

### CROSS-REFERENCE TO PRIOR APPLICATIONS

This application claims the benefit of U.S. Provisional Patent Application No. 62/095,303, filed on Dec. 22, 2014 and European Application No. 15171611.5, filed on Jun. 11, 2015. These applications are hereby incorporated by reference herein.

### BACKGROUND

1. Technical Field

The present invention generally relates to wearable technology. More specifically, the present invention relates to long term harm detection using a wearable device.

2. Description of the Related Art

Wearable electronic devices, or as used herein, wearable technology is a new class of electronic systems that can provide data acquisition through a variety of unobtrusive sensors that may be worn by a user. The sensors gather information, for example, about the environment, the user's activity, or the user's health status. However, there are significant challenges related to the coordination, computation, communication, privacy, security, and presentation of the collected data. Additionally, there are challenges related to power management given the current state of battery technology. Furthermore, analysis of the data is needed to make the data gathered by the sensors useful and relevant to end-users. In some cases, additional sources of information may be used to supplement the data gathered by the sensors. The many challenges that wearable technology presents require new designs in hardware and software.

Wearable technology may include any type of mobile electronic device that can be worn on the body, or attached to or embedded in clothes and accessories of an individual. Numerous examples of wearable technology currently exist in the consumer marketplace. Processors and sensors associated with the wearable technology can display, process or gather information. Such wearable technology has been used in a variety of areas, including monitoring health data of the user as well as other types of data and statistics. These types of devices may be readily available to the public and may be easily purchased by consumers. Examples of some wearable technology in the health arena include FitBit, Nike FuelBand, and the Apple Watch.

The Occupational Safety and Health Administration (OSHA) is an agency of the United States Department of Labor. OSHA seeks to ensure safe and healthy working conditions for men and women by setting and enforcing a variety of standards for different workplace environments. OSHA also provides training, outreach, education and assistance to further this mission. One example of ensuring safe and healthy working conditions may include enforcing standards concerning and otherwise prohibiting long-term exposure to certain types of substances, chemicals, or other environmental factors such as radiation. Consumers and workers often do not have knowledge of real-time effects of exposures nor do they have personal knowledge that such exposures are even harmful or occurring in the first place.

### SUMMARY

A first aspect of the invention includes a computer-implemented method for long term harm detection through a wearable device. The method comprises updating information relating to one or more exposures to be detected from a network, wherein the updated information includes health-based threshold limits and is stored on a wearable device; obtaining sensor data from the various sensors associated with a wearable device, wherein the sensor data relates to one or more exposures to be detected; calculating a real-time exposure level based on the obtained sensor data: evaluating whether the calculated real-time exposure level exceeds health-based threshold limits; calculating an updated long-term exposure level, wherein the calculated long-term exposure level includes combining the calculated real-time exposure level with previously stored long-term exposure level calculations; evaluating whether the calculated updated long-term exposure level exceeds health-based threshold limits; and storing the calculated updated long-term exposure level into memory of the wearable device, wherein the stored calculated long-term exposure level is used to calculate a next updated long-term exposure level.

A second aspect of the invention includes a wearable device for long term harm detection. The wearable device comprises sensors relating to one or more exposures to be detected; memory configured for storing instructions and information related to sensor detection: a network interface configured for communicatively connecting to a network: and a processor configured for updating information relating to one or more exposures to be detected from the network, wherein the updated information includes health-based threshold limits and is stored in memory of the wearable device, obtaining sensor data from the various sensors of the wearable device, calculating a real-time exposure level based on the obtained sensor data, evaluating whether the calculated real-time exposure level exceeds health-based threshold limits, calculating an updated long-term exposure level, wherein the calculated long-term exposure level includes combining the calculated real-time exposure level with previously stored long-term exposure level calculations, evaluating whether the calculated updated long-term exposure level exceeds health-based threshold limits, and storing the calculated updated long-term exposure level into memory of the wearable device, wherein the stored calculated long-term exposure level is used to calculate a next updated long-term exposure level.

The current invention aims to provide systems and methods directed at obtaining sensor data for various different exposures that a user may be exposed to, evaluating the sensor data related to the exposures that have been acquired over a period of time, and providing real-time analysis based on the evaluation of sensor data over the period of time informing the user about one or more effects that the exposures may have on the health of the user.

### BRIEF DESCRIPTION OF THE DRAWINGS

FIG. 1A illustrates an exemplar) wearable device for detection and notification of long-term exposures according to an embodiment of the present invention.

FIG. 1B illustrates exemplary sensor outputs from the wearable device relating to exposure levels over a period of time according to an embodiment of the present invention.

FIG. 2 illustrates an exemplary method for the base software of the wearable device according to an embodiment of the present invention.

FIG. 3 illustrates an exemplary system used for monitoring and notifying the user about exposure levels according to an embodiment of the present invention.

FIG. 4 illustrates an exemplary embodiment for monitoring exposure to sound according to an embodiment of the present invention.

FIG. 5 illustrates exemplary methods for the base software in the long term harm detection network according to an embodiment of the present invention.

FIG. 6 illustrates an exemplary computing device architecture that may be utilized to implement the various features and processes according to an embodiment of the present invention.

FIG. 7 illustrates an exemplary method of the present invention.

### DETAILED DESCRIPTION

The present invention is directed towards a wearable device that can be used to detect various conditions in the environment that can be harmful to the user. Some examples of environmental conditions that a user may be exposed to may include sound, radiation and airborne pollutants. Although the impact on the health of a user may not be immediately apparent, long term exposure to these various environmental conditions can arise over time with continued exposure to the same condition.

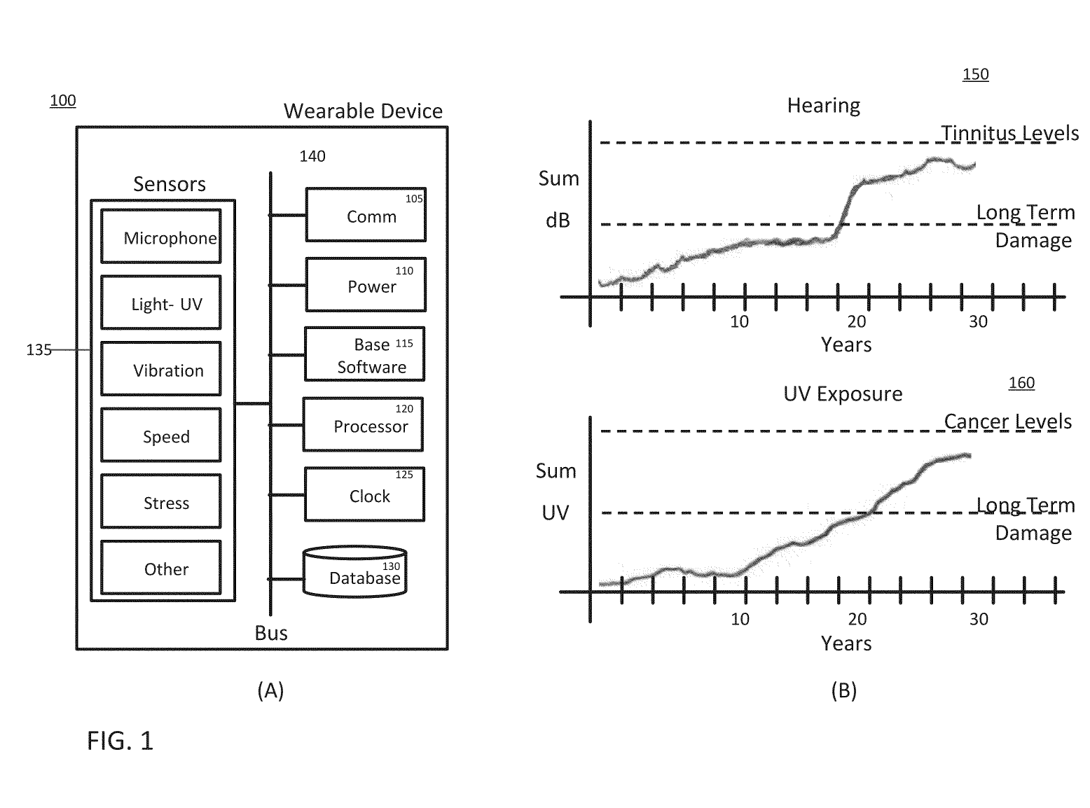

FIG. 1A illustrates an exemplary wearable device 100 for detection and notification of long-term exposures. The wearable device 100 may include a variety of different elements. These elements may include a communication module 105, a power supply 110, base software 115, a processor 120, a clock 125, memory 130 and one or more sensors 135. Each of the elements listed may be connected to a central bus 140. As used herein, the central bus 140 may be used to transfer data between the various elements of the wearable device 100. The central bus 140) may include related hardware components (e.g., wire, optical fiber) and software (e.g., communication protocols).

The communication module 105 may facilitate communication (e.g., wireless communication) between the wearable device 100 and other devices (e.g., wearable devices, smart devices) and/or networks. The communication module 105 may implement the communication through the use of one or more methods known in the art including Wi-Fi, Bluetooth, 3G, 4G, LTE, near field communication (NFC).

The power supply 110 may be included to provide power for the operation of the wearable device 100. The power supply 110 may be implemented through the use of a capacitor or a battery. The power supply 110 may also be capable of being charged or re-charged using an external power source (e.g. battery charger).

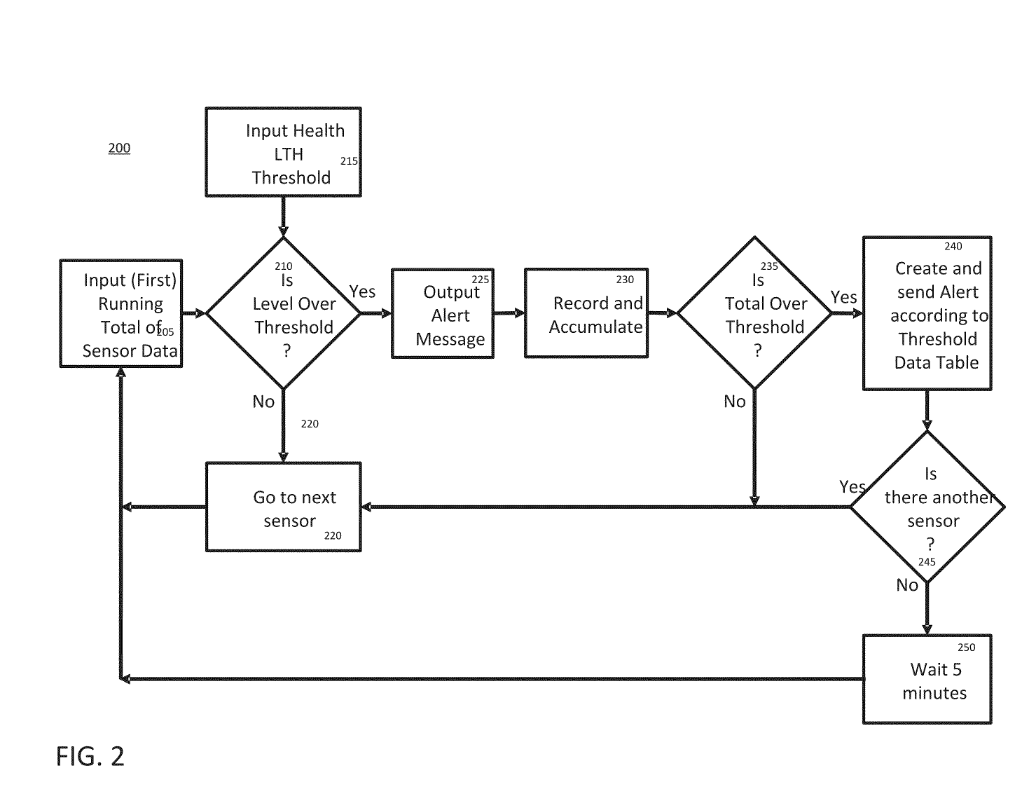

The base software 115 of the wearable device 100 may be responsible for the management and operation of the wearable device 100. In an embodiment, the base software 115 may poll for sensor data relating to an exposure level for the user. The base software may also execute software and other elements within the wearable device 100 to carry out the functionality of the wearable device 100. Further discussion of the base software can be seen below (see FIG. 2).

The processor 120 of the wearable device 100 may be any computer processor known in the art. The processor 120 can be used to carry out the various instructions of the wearable device 100 (e.g., analysis of sensor data, calculations). In some embodiments, the wearable device 100 may include two or more processors.

The wearable device 100 may also include a clock 125. The clock 125 may be used to provide time-based data that can be used by the wearable device 100. For example, the wearable device 100 may use the clock 125 to provide a time-stamp (e.g., date, time of day) for sensor data obtained by the sensors 135 of the wearable device 100. Since the present invention is directed at analyzing long-term exposure levels of a user, the clock 125 may be helpful in organizing and providing context for the sensor data obtained by the sensors 135.

The memory 130 included in the wearable device 100 may include one or more databases. The memory 130 may be used to store various data within the wearable device. For example, the memory 130 may be used to store sensor data obtained by the sensors 135. The memory 130 may also be used to store outputs (e.g., calculations) created by the processor 120 based on instructions received from the base software 115.

The wearable device 100 may include one or more sensors 135. In particular, the sensors 135 may be directed at measuring different parameters in the environment relating to exposure levels. As illustrated in the figure, the sensors may include sensors for detecting sound (e.g., microphone), ultra-violet (UV) light or other forms of radiation such as infrared, vibrations, speed, and stress. It should be noted that more or less sensors may be used in a wearable device. Other types of sensors not be listed above may also be included in the wearable device. Some examples of other sensors may include sensors that can be used to measure biometric parameters (e.g., blood pressure, pulse, temperature) of the user. In an embodiment, the wearable device may be able to monitor various biometric parameters in connection with detected exposure levels. The sensor data corresponding to the biometric parameters may also be used to evaluate the health condition of the user in view of the detected exposure.

FIG. 1B illustrates exemplary sensor outputs from the wearable device relating to exposure levels over a period of time. In particular, the two graphical displays 150, 160 illustrate different outputs from sensors used to detect sound 150 and UV exposure 160. The graphs may provide a summary of sensor data obtained over a period of time for each respective exposure. As illustrated in the graphical displays, each of the sensor data can be displayed for periods over many years (e.g., 30+ years). In an embodiment, these graphical displays may be provided to the user for purposes of informing the user of exposure levels and associated impact on the health condition of the user.

The graphical displays 150, 160 may also provide one or more health-based threshold limits. The health-based threshold limits can be used to identify when an amount of exposure is negatively impacting the user as some levels of exposure to a particular exposure-type may be normal or acceptable. It should be noted that different environmental conditions may have different health-based threshold limits indicating normal and negative exposure levels.

Over time, however, negative effects (e.g., long-term damage, increased possibility of cancer) may arise due to the accumulated exposure levels. For example, as seen in the graphical display for UV exposure 160, after 22 years of being exposed to UV radiation, the wearable device may have informed the user that long term damage has occurred. Presuming that the exposure to UV radiation does not change (e.g., minimized or eliminated) after the user has been exposed to UV radiation for over 30 years, the user may have a higher possibility of cancer. The health-based threshold limits can be used as a measure to inform the user when precautions may be necessary to minimize or eliminate further exposure in an attempt to minimize or eliminate further long term negative impacts based on the exposure.

FIG. 2 illustrates an exemplary method 200 for the base software of the wearable device. As noted above, the base software of the wearable device (as illustrated in FIG. 1), may be used to poll for sensor data related to one or more environmental conditions that the user is being exposed to from the various sensors associated with the wearable device.

In step 205 of FIG. 2, the base software inputs a running total of sensor data obtained by one of the sensors associated with the wearable device. A selected sensor (e.g., microphone) may be used to measure parameters for a particular environmental condition (e.g., sound levels). As noted above, the wearable device may include numerous sensors that can be directed towards a distinct environmental condition. In an embodiment, two or more sensors may obtain sensor data that can be used to evaluate exposure levels of a single environmental condition. In any case, the sensor data may also be taken at regular intervals (e.g., hourly, daily, weekly).

The running total of sensor data obtained by the base software includes the most current sensor data obtained by the corresponding sensor. The running total can refer to the sensor data obtained by the base software in between execution of the evaluation of the current sensor data by the base software. For example, in an embodiment, sensor data may be taken at regular intervals (e.g., every minute, 5 minutes). The base software, however, may not perform an evaluation of the current sensor data along similar intervals but rather may have multiple sensor data in queue to be evaluated together.

In step 210, the base software can evaluate the running total for the sensor data with respect to one or more health-based threshold limits (as illustrated in FIG. 1B). The evaluation can determine if current obtained sensor data readings relating to exposure level to a particular environment condition may currently pose a health-based concern to the user.

It should be noted that the health-based threshold limits may be provided for use by the wearable device by a variety of different sources 215. For example, organizations (e.g., OSHA) may provide healthful working standards that can be incorporated into the health-based threshold limits. Environment-based organizations that study a particular environmental condition may also be capable of providing health-based threshold limits that the user can use with the wearable device. In other embodiments, these health-based threshold limits may be downloaded from the cloud or Internet and stored into memory of the wearable device for use by the base software.

Based on the evaluation performed by the base software in step 210, the base software can proceed along two different branches 220, 225. If the base software determines that the evaluated sensor data correspond to acceptable exposure levels for the environmental condition, the base software may then proceed with the next sensor that has not been evaluated 220. In step 220, the base software can then determine what sensors have not yet been evaluated during a current iteration and load a running total of sensor data corresponding to that new sensor (see step 205). If the evaluated sensor data, however, is over a health-based threshold limit, the base software may provide an alert to the user 225. The alert may be used to inform the user that a current exposure level to current environmental conditions may be unhealthy or dangerous.

The alert (or notification) may be provided in many ways. For example, information about the exposure level and possible negative effects may be displayed on the wearable device. The wearable device may also output a signal (e.g., sound, vibration) that can be used to signal that exposure levels for an environmental condition has exceeded acceptable levels. In some embodiments, information (e.g., suggestions) relating to minimizing or eliminating additional exposure to a particular environmental condition may also be provided as a preventative measure.

In step 230 of FIG. 2, the base software records the running total of the recently obtained sensor data acquired from the sensor of the wearable device and combines the recently obtained sensor data with the past sensor data previously obtained. In particular, over multiple iterations of the base software, the accumulation of recently obtained sensor data with past sensor data can be used to provide a continually growing summary of exposure to a particular environmental condition over a period of time. The running total can be indicative of long-term exposure for the user related to the particular environmental condition. It should be noted that the past data may be stored, for example, in memory associated with the wearable device.

In step 240 of FIG. 2, an evaluation is performed based on the accumulated sensor data relating to exposure levels. The evaluation may be performed in a similar manner as in step 210. As noted above, health-based threshold limits may be provided that can be used to compare the exposure levels for the user. These health-based thresholds may be different than the ones provided above in step 210 since these limits take into account exposure for longer periods of time (e.g., years).

Based on the evaluation in step 235, if an accumulated total exposure is still considered acceptable (e.g., below the health-based threshold limit), the base software may proceed to the next sensor (see step 220). The base software may choose a different sensor to upload corresponding running total sensor data to be evaluated (see step 205).

If the accumulated total exposure, however, is over the health-based threshold limit, in step 240 of FIG. 2, the base software may create and send an alert. The alert may include a notification (e.g., signal, message) that can be used to inform the user that a long-term exposure to a particular environmental condition poses negative effects on the health condition of the user. The base software may perform similar processes in step 240 as described above in step 225.

Once notification has been provided to the user, the base software can determine whether other sensors associated with the wearable device have sensor data that have not been evaluated in a current iteration. In particular, in step 245 of FIG. 2, the base software determines if further evaluations need to be performed for other sensors. For each sensor associated with the wearable device, the base software may execute steps 205-240 to evaluate exposure levels of the user and determine if the exposure levels negatively impact the health condition of the user.

Once all the sensors (and corresponding running totals of sensor data) have been evaluated, the base software may wait a pre-defined period of time (e.g., 5 minutes) before performing the steps of the method 200 described again for the sensors associated with the wearable device. In this way, the user may be kept up-to-date as to the current effects of being exposed to various environmental conditions. The method 200 of the base software also continually acquires sensor data that can be used to evaluate effects of being exposed to various environmental conditions for long term periods.

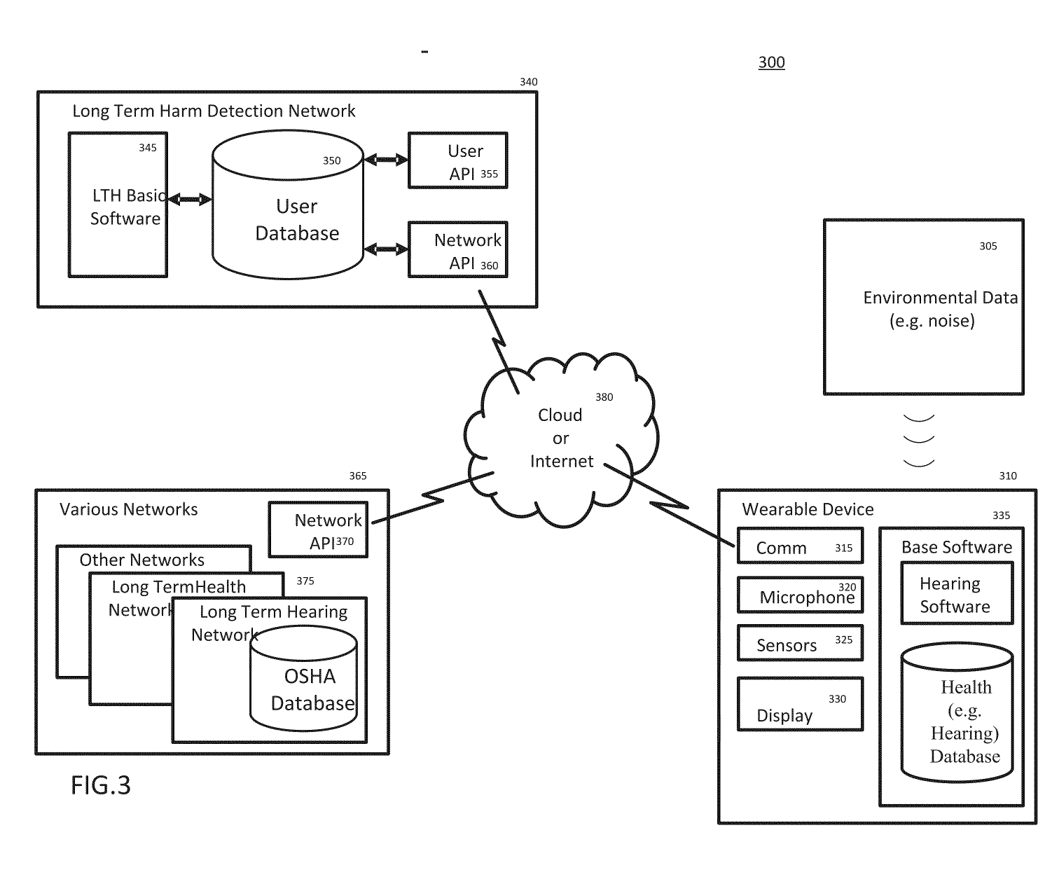

FIG. 3 illustrates an exemplary system used for monitoring and notifying the user about exposure levels. In particular, the figure illustrates an embodiment of the system 300 directed towards monitoring sound 305.

As illustrated in FIG. 3, the system 300 includes a wearable device 310, a long term harm detection network 340 and various other networks 365. The wearable device 310 may be used to measure and evaluate sensor data obtained about an environmental condition 305, which in this case is sound. It should be noted that the wearable device 310, the long term harm detection network 340 and the various other networks 355 may all be connected to the cloud or Internet 380.

The wearable device 310 may be similar to the wearable device 100 of FIG. 1, described above. As illustrated in FIG. 3, the wearable device 310 may contain similar elements including a communication module 315, sensors (320, 325) and base software 335. Included in the wearable device 310 that is distinct from the wearable device 100 of FIG. 1 may be a display 330. The display 330 may be used to provide notification (e.g., messages) and data regarding a particular exposure. In some embodiments, the display 330 may also be a touch screen display that may allow the user to interact with the wearable device. In other embodiments, it should be noted that the wearable device 100 of FIG. 1 may also contain a display for similar reasons.

With the embodiment illustrated in FIG. 3 directed at detecting sound-based exposure, the wearable device 310 may also contain additional software and databases associated with the particular environmental condition to be monitored. In particular, a hearing software and database may be included in the base software 335 to facilitate the detection of sound-based exposure and notification of the health condition of the user based on the sound-based exposure. It should be noted that other types of exposures may also be incorporated using the system 300 of FIG. 3. In these other embodiments, the base software may also include software and a database corresponding to the other types of exposures. These databases for each exposure type may be separate. In another embodiment, all exposure-related data may be stored in a single database with identification used to distinguish the sensor data apart from other sensor data of different exposure types.

As illustrated in FIG. 3, the system 300 also includes the long term harm detection network 340. The network 340 may include information that can be used by the wearable device 310 of the user in evaluating sensor data to determine whether long term harm has occurred for one or more exposures.

The long term harm detection network 340 as illustrated in FIG. 3 includes its own base software 345, memory 350 (e.g. database) and application programming interfaces (APIs). The base software 345 of the long term harm detection network 340 may be used to manage and operate the particular network 340. For example, the base software 345 may be used to obtain information from other networks 365 or from the wearable device 310 of the user. The memory may be used to store information used by the long term harm detection network 340. Exemplary information may include health-based threshold limits and messages/warnings that can be provided to the user if a corresponding health-based threshold limit was exceeded. The APIs 355, 360 may be used to connect the long term harm detection network 340 with the wearable device 310 of the user or other networks 365, respectively. Communication can be performed through the cloud or Internet 380.

The various other networks 365 shown in FIG. 3 may be a collection of various networks related to detection and notifying the user of a particular exposure-related concern. As illustrated in FIG. 3, the various networks 365 may include networks that may include information (e.g., health-based threshold limits) from third parties (e.g., OSHA) that can be used to evaluate sensor data. The information may be accessed by the user through the wearable device 310. In some embodiments, other networks may search for information from the various networks to populate their database with necessary information. For example, it may be possible that the long term harm detection network 340 can search the various networks 365 for information stored in one or more of the networks (e.g., OSHA database) to be downloaded and stored into memory 350. In any case, networks and/or the user can access the various networks 365 through the use of the network API 370 associated with the various networks 365.

In one embodiment, the system 300 may be initialized by having the various networks 365 (e.g. long term hearing network 375) load information stored on the various networks (e.g., OSHA standards pertaining to sound-based exposures) into the long term health detection network 340 using the network API 360, 370. In particular, the information may be stored into memory 350 of the long term health detection network 340. Afterwards, the long term harm detection network 340 may begin polling for information requests from one or more users through their respective wearable devices 310.

When one or more user requests are received by the long term harm detection network 340, the base software 345 of the network 340 may load the requested information (e.g., OSHA databases) into appropriate databases found in the wearable device 310. The base software 355 of the wearable device 310 can then be executed to obtain sensor data about an exposure (e.g., noise) from the various sensors 320, 325. As noted above, sensor data may be obtained at regular intervals of time (e.g., 5 minutes), accumulated over different periods of time (e.g., daily, weekly, monthly) and stored in the wearable device 310. The base software 335 of the wearable device 310 may also evaluate sensor data and the accumulated long-term totals with the health-based threshold limits. These health-based threshold limits may have been included, for example, in the information downloaded from the long term harm detection network 340 and/or provided by the various networks 365.

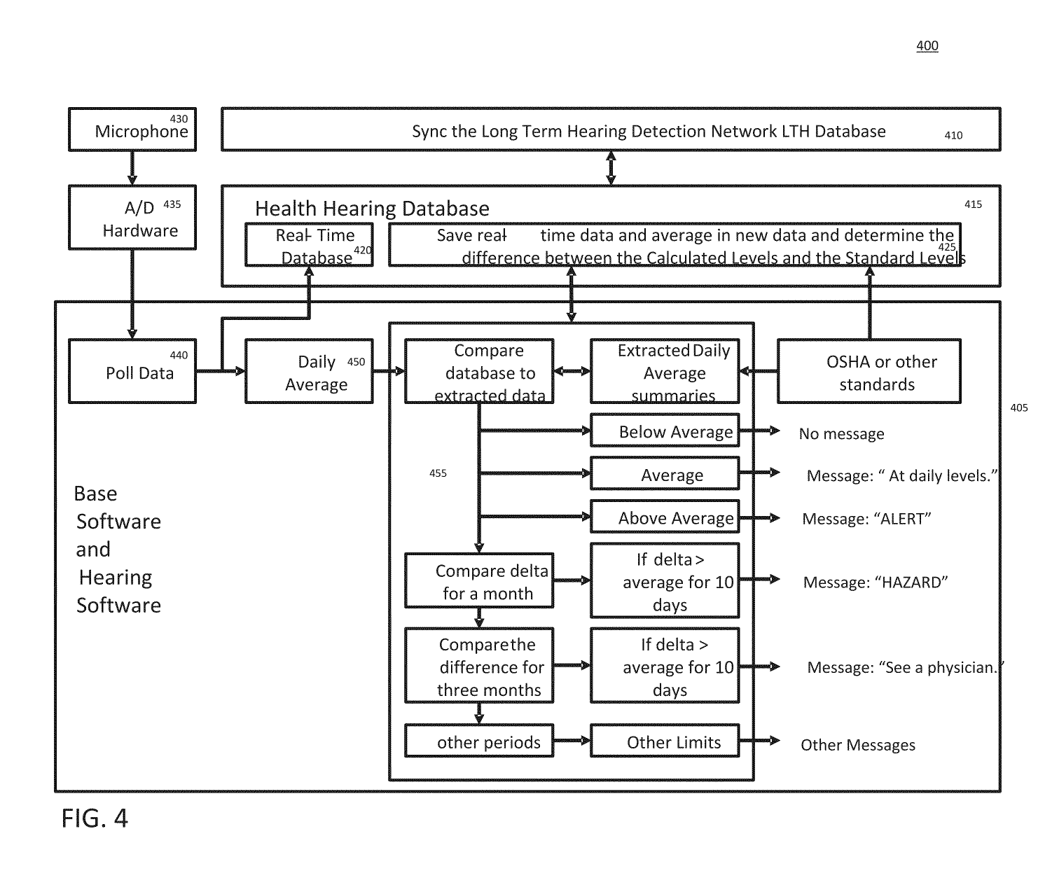

FIG. 4 illustrates an exemplary embodiment for monitoring exposure to sound. As illustrated in FIG. 4, base software and exposure-related software 405 are included in the wearable device. The base software and exposure-related software 405 may be similarly found in FIG. 3 and discussed in corresponding sections above.

As shown in FIG. 4, the wearable device first syncs the information stored in the long term harm detection network with the information stored in memory of the wearable device (410). As noted above, the long term harm detection network may include information from various networks that can be used to monitor and/or detect exposure levels for different types of environmental conditions. The wearable device may be able to download particular sets of information directed at different exposures that the user would like to monitor.

With respect to the memory (e.g., health hearing database) of the wearable device 415, the wearable device may include separate databases to store real-time sensor data obtained by the various sensors 420 associated with the wearable device and long-term combined/accumulated data 425.

The real-time database may be a list of recently obtained sensor data obtained from one of the sensors 430 associated with the wearable device. In the example, as illustrated in FIG. 4, the sensor 430 may be a microphone. The sensor data may then be converted (e.g. Audio-to-Digital converter hardware 435) into a form that can be processed by the wearable device. The sensor data may be obtained by the base software 405 at regular intervals (440). In particular, the base software 405 may poll the sensors for sensor data and store the obtained sensor data into the real-time database 420.

The real-time sensor data obtained by the base software 405 may then be evaluated. The sensor data obtained by the base software 405 may be operated on (e.g., averaged) with past data (450). Afterwards, the base software 405 may perform various evaluations/comparisons 455. For example, comparisons between the real-time data and accumulated data can then be performed (e.g., delta) with the information downloaded from the long term harm detection network (e.g. OSHA standard) to quantify whether the user has been exposed to harmful levels of exposure. These comparisons can also be performed over particular periods of time. The user can then be informed accordingly based on a difference between an acceptable threshold level and what was actually measured and/or calculated. For example, the user may be provided a message if a health-based threshold limit has been exceeded. The comparisons are then saved in the database 425 for future use. It should be noted that the long term data, generated by combining/accumulating recently obtained sensor data with the long term data, may also be stored in the database 425 for future use.

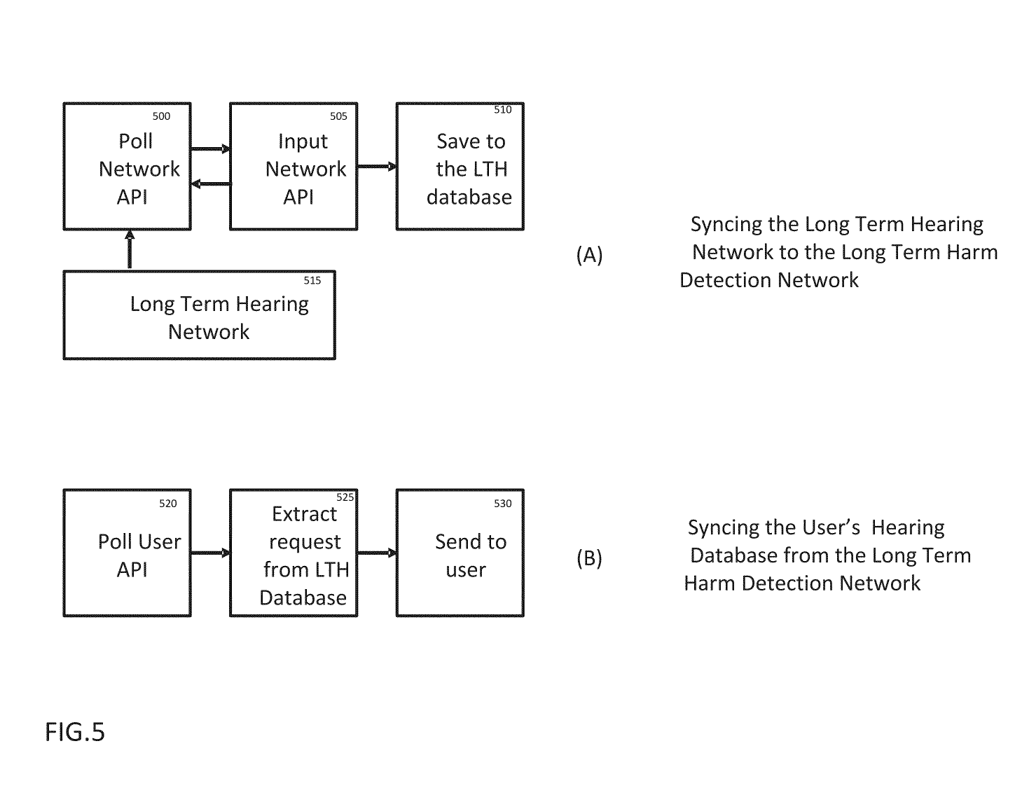

FIG. 5 illustrates exemplary methods for the base software in the long term harm detection network. As noted above, the base software for the long term harm detection network may be used to manage and operate the long term harm detection network. As described below, the base software may be responsible for acquiring necessary information from various networks to be stored on the long term harm detection network. The base software may also be responsible for responding to user requests.

FIG. 5A shows an exemplary method of syncing information stored in various networks (e.g., the long term hearing network) with the database of the long term harm detection network. As noted above, the long term harm detection network may be required to look for information from various other networks that may be helpful for the user in evaluating whether an exposure level is harmful.

In step 500, the base software of the long term harm detection network polls the network API for a connection with one or more networks from the available various networks (e.g., long term hearing network 515). The polling may include the base software of the long term harm detection network sending out a request to the various networks for any available information related to a particular exposure (e.g., sound).

In step 505, the base software of the long term harm detection network then takes as input the information provided by one or more of the various networks connected with the long term harm detection network using the network API. The information that is being inputted by the base software may include requested information that a user has requested pertaining to a particular exposure. In other embodiments, the long term harm detection network may seek to update its information stored within the network on a regular basis and the information being inputted may be information that is new.

In step 510, the base software stores the information obtained from the various networks into memory of the long term harm detection network. It should be noted that the various networks may provide the requested information as specified by the long term harm detection network. In other embodiments, the various networks may provide their entire database for the base software to evaluate whether any information included is new. In any case, the information that was previously not included in the memory of the long term harm detection network can then be stored so that users may be able to acquire such information if desired.

FIG. 5B illustrates an exemplary method whereby the long term harm detection network can sync information stored within the memory of the network with the memory of the wearable device of the user. This method may facilitate providing the user with information (e.g., health-based threshold limits) that can be used to evaluate sensor data related to exposure levels. This method may be executed based on a request for information about a particular exposure.

In step 520, the long term harm detection network polls the user API. The polling may allow the long term harm detection network to determine if a request is being provided by one or more users through the use of the wearable device.

Upon receipt of a request for information stored within the long term harm detection network, in step 525, the long term harm detection network can search the memory associated with the network and obtain the requested information. In some embodiments, the request may relate to a particular type of exposure (e.g., sound). This group of information can then be provided to the user. In other embodiments, the request may provide a request for any new or up-to-date information based on a last time the wearable device was last updated. The long term harm detection network may be able to search for any applicable information after the date of the last update and provide the requested information to the user.

In step 530, the information that was requested by the user can then be provided from the long term harm detection network (via the user API) to the wearable device of the user. The information can then be stored in the memory of the wearable device and usable for the various evaluations and processes of the wearable device.

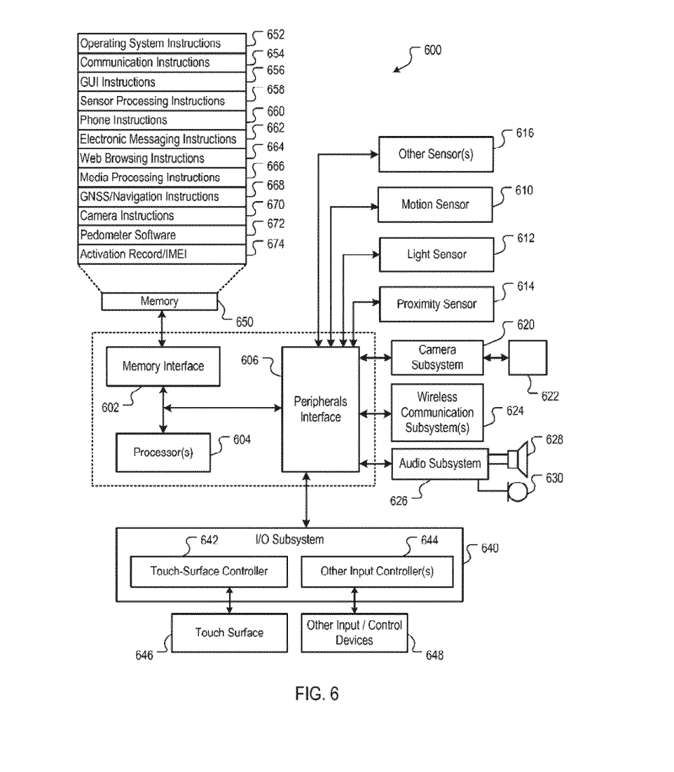

FIG. 6 illustrates an exemplary computing device architecture that may be utilized to implement the various features and processes described herein. For example, the computing device architecture 600 could be implemented in a pedometer. Architecture 600 as illustrated in FIG. 6 includes memory interface 602, processors 604, and peripheral interface 606. Memory interface 602, processors 604 and peripherals interface 606 can be separate components or can be integrated as a part of one or more integrated circuits. The various components can be coupled by one or more communication buses or signal lines.

Processors 604 as illustrated in FIG. 6 is meant to be inclusive of data processors, image processors, central processing units, or any variety of multi-core processing devices. Any variety of sensors, external devices, and external subsystems can be coupled to peripherals interface 606 to facilitate any number of functionalities within the architecture 600 of the exemplar mobile device. For example, motion sensor 610, light sensor 612, and proximity sensor 614 can be coupled to peripherals interface 606 to facilitate orientation, lighting, and proximity functions of the mobile device. For example, light sensor 612 could be utilized to facilitate adjusting the brightness of touch surface 646. Motion sensor 610, which could be exemplified in the context of an accelerometer or gyroscope, could be utilized to detect movement and orientation of the mobile device. Display objects or media could then be presented according to a detected orientation (e.g., portrait or landscape).

Other sensors could be coupled to peripherals interface 606, such as a temperature sensor, a biometric sensor, or other sensing device to facilitate corresponding functionalities. Location processor 615 (e.g., a global positioning transceiver) can be coupled to peripherals interface 606 to allow for generation of geo-location data thereby facilitating geo-positioning. An electronic magnetometer 616 such as an integrated circuit could be connected to peripherals interface 606 to provide data related to the direction of true magnetic North whereby the mobile device could enjoy compass or directional functionality. Camera subsystem 620 and an optical sensor 622 such as a charged coupled device (CCD) or a complementary metal-oxide semiconductor (CMOS) optical sensor can facilitate camera functions such as recording photographs and video clips.

Communication functionality can be facilitated through one or more communication subsystems 624, which may include one or more wireless communication subsystems. Wireless communication subsystems 624 can include 802.x or Bluetooth transceivers as well as optical transceivers such as infrared. Wired communication subsystems can include a port device such as a Universal Serial Bus (USB) port or some other wired port connection that can be used to establish a wired coupling to other computing devices such as network access devices, personal computers, printers, displays, or other processing devices capable of receiving or transmitting data. The specific design and implementation of communication subsystem 624 may depend on the communication network or medium over which the device is intended to operate. For example, a device may include wireless communication subsystem designed to operate over a global system for mobile communications (GSM) network, a GPRS network, an enhanced data GSM environment (EDGE) network, 802.x communication networks, code division multiple access (CDMA) networks, or Bluetooth networks. Communication subsystem 624 may include hosting protocols such that the device may be configured as a base station for other wireless devices. Communication subsystems can also allow the device to synchronize with a host device using one or more protocols such as TCP/IP, HTTP, or UDP.

Audio subsystem 626 can be coupled to a speaker 628 and one or more microphones 630 to facilitate voice-enabled functions. These functions might include voice recognition, voice replication, or digital recording. Audio subsystem 626 in conjunction may also encompass traditional telephony functions.

I/O subsystem 640 may include touch controller 642 and/or other input controller(s) 644. Touch controller 642 can be coupled to a touch surface 646. Touch surface 646 and touch controller 642 may detect contact and movement or break thereof using any of a number of touch sensitivity technologies, including but not limited to capacitive, resistive, infrared, or surface acoustic wave technologies. Other proximity sensor arrays or elements for determining one or more points of contact with touch surface 646 may likewise be utilized. In one implementation, touch surface 646 can display virtual or soft buttons and a virtual keyboard, which can be used as an input/output device by the user.

Other input controllers 644 can be coupled to other input/control devices 648 such as one or more buttons, rocker switches, thumb-wheels, infrared ports, USB ports, and/or a pointer device such as a stylus. The one or more buttons (not shown) can include an up/down button for volume control of speaker 628 and/or microphone 630. In some implementations, device 600 can include the functionality of an audio and/or video playback or recording device and may include a pin connector for tethering to other devices.

Memory interface 602 can be coupled to memory 650. Memory 650 can include high-speed random access memory or non-volatile memory such as magnetic disk storage devices, optical storage devices, or flash memory. Memory 650 can store operating system 652, such as Darwin, RTXC, LINUX, UNIX, OS X, ANDROID, WINDOWS, or an embedded operating system such as VxWorks. Operating system 652 may include instructions for handling basic system services and for performing hardware dependent tasks. In some implementations, operating system 652 can include a kernel.

Memory 650 may also store communication instructions 654 to facilitate communicating with other mobile computing devices or servers. Communication instructions 654 can also be used to select an operational mode or communication medium for use by the device based on a geographic location, which could be obtained by the GPS/Navigation instructions 668. Memory 650 may include graphical user interface instructions 656 to facilitate graphic user interface processing such as the generation of an interface; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GPS/Navigation instructions 668 to facilitate GPS and navigation-related processes, camera instructions 670 to facilitate camera-related processes and functions; and instructions 672 for any other application that may be operating on or in conjunction with the mobile computing device. Memory 650 may also store other software instructions for facilitating other processes, features and applications, such as applications related to navigation, social networking, location-based services or map displays.

Each of the above identified instructions and applications can correspond to a set of instructions for performing one or more functions described above. These instructions need not be implemented as separate software programs, procedures, or modules. Memory 650 can include additional or fewer instructions. Furthermore, various functions of the mobile device may be implemented in hardware and/or in software, including in one or more signal processing and/or application specific integrated circuits.

Certain features may be implemented in a computer system that includes a back-end component, such as a data server, that includes a middleware component, such as an application server or an Internet server, or that includes a front-end component, such as a client computer having a graphical user interface or an Internet browser, or any combination of the foregoing. The components of the system can be connected by any form or medium of digital data communication such as a communication network. Some examples of communication networks include LAN, WAN and the computers and networks forming the Internet. The computer system can include clients and servers. A client and server are generally remote from each other and typically interact through a network. The relationship of client and server arises by virtue of computer programs running on the respective computers and having a client-server relationship to each other.

One or more features or steps of the disclosed embodiments may be implemented using an API that can define on or more parameters that are passed between a calling application and other software code such as an operating system, library routine, function that provides a service, that provides data, or that performs an operation or a computation. The API can be implemented as one or more calls in program code that send or receive one or more parameters through a parameter list or other structure based on a call convention defined in an API specification document. A parameter can be a constant, a key, a data structure, an object, an object class, a variable, a data type, a pointer, an array, a list, or another call. API calls and parameters can be implemented in any programming language. The programming language can define the vocabulary and calling convention that a programmer will employ to access functions supporting the API. In some implementations, an API call can report to an application the capabilities of a device running the application, such as input capability, output capability, processing capability, power capability, and communications capability.

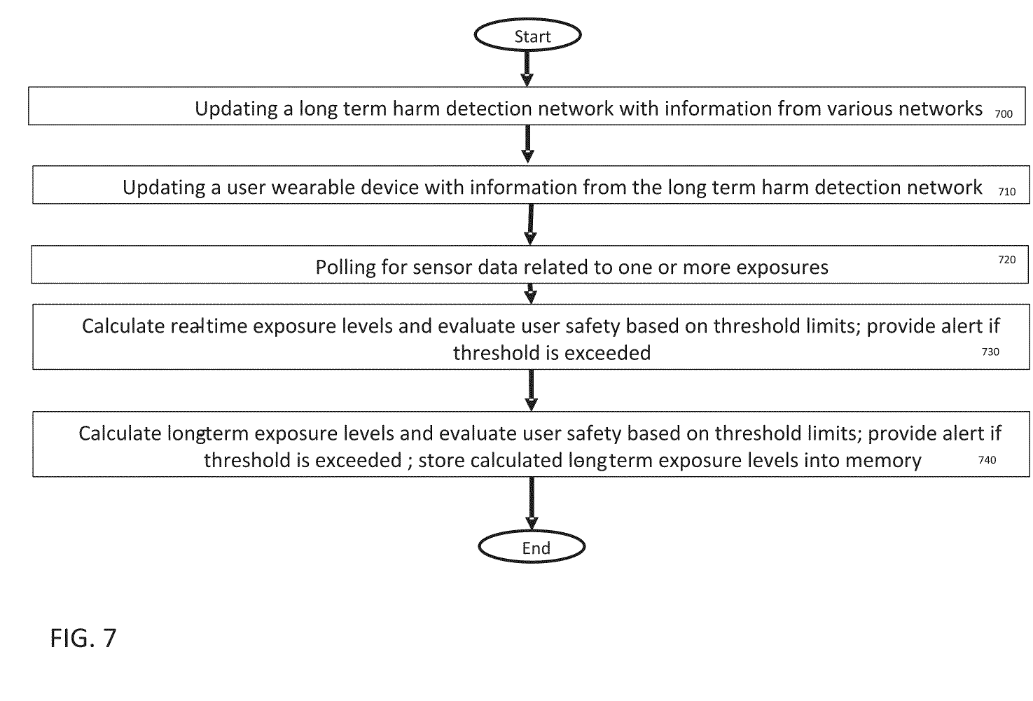

FIG. 7 illustrates an exemplary method of the present invention. As discussed above, the present invention may be directed at detecting exposures levels and determining whether the detected exposure levels are negatively affecting the health condition of the user. If detected exposure levels exceed acceptable thresholds, an alert can be provided to the user.

In step 700, the long term harm detection network may be updated with related information from the various networks. The long term harm detection network may be a network that compiles various types of information that a user can utilize to evaluate long term exposure levels.

The user, upon requesting information for a particular exposure level, can send a request to update the information stored in the wearable device. In step 710, for example, if the information already exists in the long term harm detection network, the information can be provided to the user. If the information does not exist, however, the long term harm detection network may search the various networks for the requested information. Once the new information is obtained by the long term harm detection network, the information can then be relayed back to the user. In some embodiments, the long term harm detection network may periodically update the information stored in the long term harm detection network to ensure the most up-to-date information can be provided to the user.

In step 720, the wearable device of the user can then obtain sensor data pertaining to exposure levels for one or more different environmental conditions (e.g., sound, UV radiation, pollution) that can negatively affect the health condition of the user. The sensor data may be obtained at regular intervals (e.g., 5 minutes) to ensure that up-to-date information regarding the health condition of the user can be evaluated. The polled sensor data may be stored in memory of the user wearable device.

In step 730, the wearable device (via instructions from the base software) may calculate real-time exposure levels using the polled sensor data. The calculated real-time exposure levels can then be compared with the health-based threshold limits to determine whether a particular exposure level of the environmental condition is, for example, safe/acceptable. In some situations, the calculated real time exposure level may exceed the threshold limit. In these situations, an alert may be provided to the user. If the exposure level is acceptable, then the base software may not provide any notification to the user.

In step 740 of FIG. 7, the wearable device calculates long-term exposure levels using the polled sensor data and previously stored long-term exposure level calculations. The calculated long-term exposure levels are similarly compared with health-based threshold limits to evaluate whether the calculated exposure levels are acceptable. In an embodiment, if the threshold limit is exceeded, the user may be provided an alert notifying the user of the issue.

After the evaluation, the long-term exposure levels can then be stored in memory for future use. As noted above, even though a current exposure to an environmental condition may not immediately affect the health condition of the user, continued exposure may also cause negative effects. The use of the calculated long-term exposure levels facilitate in detecting and determining the impact that long-term exposure to various environmental conditions may have on the user.

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen in order to best explain the principles of the technology and its practical application to thereby enable others skilled in the art to best utilize the technology in various embodiments and with various modifications as are suited to the particular use contemplated. It is intended that the scope of the technology be defined by the claim.

1. A computer-implemented method for long term harm detection through a wearable device, the method comprising:
updating information relating to one or more exposures to be detected from a network, wherein the updated information includes health-based threshold limits and is stored on a wearable device;
obtaining sensor data from the various sensors associated with a wearable device, wherein the sensor data relates to one or more exposures to be detected;
calculating a real-time exposure level based on the obtained sensor data;
evaluating whether the calculated real-time exposure level exceeds health-based threshold limits;
calculating an updated long-term exposure level, wherein the calculated long-term exposure level includes combining the calculated real-time exposure level with previously stored long-term exposure level calculations;
evaluating whether the calculated updated long-term exposure level exceeds health-based threshold limits; and
storing the calculated updated long-term exposure level into memory of the wearable device, wherein the stored calculated long-term exposure level is used to calculate a next updated long-term exposure level.

2. The method of claim 1, wherein the detected exposures include sound and UV radiation.

3. The method of claim 1, further comprising notifying a user of the wearable device of unsafe exposure levels based on evaluations whether the calculated exposure levels exceed health-based threshold limits.

4. The method of claim 3, wherein the notifying is selected from the group consisting of: (a) providing an alert and corresponding message for the user on the wearable device and (b) providing a graphical representation identifying exposure levels of the user along with corresponding health-based threshold limits.

5. The method of claim 1, wherein the obtained sensor data is obtained at regular intervals.

6. The method of claim 1, wherein the health-based threshold limits are limits defined by third party networks.

7. The method of claim 1, wherein the sensors include at least one of sensors for detecting sound, sensors for detecting ultraviolet light, sensors for detecting vibrations, and sensors for detecting speed, and sensors for detecting biometric parameters of the user.

8. A wearable device for long term harm detection comprising:
sensors relating to one or more exposures to be detected;
memory configured for storing instructions and information related to sensor detection;
a network interface configured for communicatively connecting to a network; and
a processor configured for:
updating information relating to one or more exposures to be detected from the network, wherein the updated information includes health-based threshold limits and is stored in memory of the wearable device,
obtaining sensor data from the various sensors of the wearable device,
calculating a real-time exposure level based on the obtained sensor data,
evaluating whether the calculated real-time exposure level exceeds health-based threshold limits,
calculating an updated long-term exposure level, wherein the calculated long-term exposure level includes combining the calculated real-time exposure level with previously stored long-term exposure level calculations,
evaluating whether the calculated updated long-term exposure level exceeds health-based threshold limits, and
storing the calculated updated long-term exposure level into memory of the wearable device, wherein the stored calculated long-term exposure level is used to calculate a next updated long-term exposure level.

9. The wearable device of claim 8, wherein exposures to be detected include at least one of sound and UV radiation.

10. The wearable device of claim 8, wherein the processor is configured for executing further instructions to notify the user of unsafe exposure levels based on evaluations whether the calculated exposure levels exceed health-based threshold limits.

11. The wearable device of claim 10, wherein the notification is selected from the group consisting of: (a) an alert and corresponding message for the user on the wearable device, and (b) a graphical representation identifying exposure levels of the user along with corresponding health-based threshold limits, the graphical representation provided on a graphical display of the user device.

12. The wearable device of claim 8, wherein the obtained sensor data is obtained at regular intervals.

13. The wearable device of claim 8, wherein the health-based threshold limits are limits defined by third party) networks.

14. The wearable device of claim 14, wherein the sensors include sensors selected from the group including sensors for detecting one or more of sound, ultra-violet light, vibrations, speed, stress, and biometric parameters of the user.
